(19) United States
(12) Patent Application Publication
(10) Pub. No.: US 2018/0228186 A1
(43) Pub. Date: Aug. 16, 2018

(54) METHOD OF NEUTRALIZING AMMONIA ODOR AND FLAVOR FROM SHARK AND OTHER UREA-CONTAINING FISH

(71) Applicant: High Liner Foods, Inc., Portsmouth, NH (US)

(72) Inventors: Joseph A. Galetti, Wareham, MA (US); Logan W. Pierce, Harrisburg, NC (US)

(21) Appl. No.: 15/897,215

(22) Filed: Feb. 15, 2018

Related U.S. Application Data
(60) Provisional application No. 62/459,105, filed on Feb. 15, 2017.

(51) Int. Cl. A23L 5/20; A23L 27/00; A23L 17/00; A61L 2/18; A61L 2/23

(52) U.S. Cl. CPC A23L 5/276; A23L 27/84; A23V 2002/00; A61L 2/18; A61L 2/23; A23L 17/00

(57) ABSTRACT

A composition for and method of neutralizing ammonia odor and flavor from shark and other urea-containing fish includes soaking the fish in a refrigerated (4° C.) bath containing water (88.0%), dry skim milk solids (7.5%), dry buttermilk solids (2.5%), and salt (2.0%) for 18-20 minutes, with the objective of improving consumer perception of odor and taste during consumption of the fish.

### CROSS-REFERENCE TO RELATED APPLICATIONS

This application claims the benefit of U.S. provisional patent application Ser. No. 62/459,105 filed Feb. 15, 2017.

### FIELD OF THE INVENTION

The present invention relates to the field of food processing, and specifically describes the interactions of food additives on the protein chemistry of urea-containing fish.

### BACKGROUND OF THE INVENTION

This section provides background information related to the present disclosure which is not necessarily prior art.

Shark, and other cartilaginous fish within the chondrichthyes class (rays, skates, sawfish, and chimaeras), utilize urea and other nitrogenous organic compounds within their tissues as important chemical osmoregulators. Osmoregulation is a vital process which maintains osmotic pressure between cell walls through the control of fluid and electrolytes, which prevents cellular disruption. Additionally, sharks lack traditional urinary systems or swim bladders, therefore they concentrate urea in their blood and excrete it through their skin.

The buildup of urea in the flesh of shark is rapidly converted into ammonia, trimethylamine, and other nitrogenous breakdown products post-mortem. The ammonia compounds present in the flesh generally result in consumer rejection during consumption, due to perceived off-odors and flavors. Therefore, attempts have been made to neutralize, mask, or remove these ammonia compounds from sharks and other urea-containing fish.

Soaking shark meat in buttermilk has traditionally been the technique of choice employed by chefs to neutralize and mask the strong ammonia flavor of the flesh. The two compounds within buttermilk that contribute to this effect are casein and lactic acid. Casein is the predominant protein type found in mammalian milk, and has the ability to bind with and extract the nitrogenous compounds found within the flesh of shark. Lactic acid is the primary sugar present in milk products, and neutralizes these nitrogenous compounds by chemical breakdown. Additionally, the fat content of buttermilk masks off-flavors associated with said compounds.

Another popular technique to mask the ammonia flavor of shark, and other urea-containing fish, involves soaking the fish in a water bath containing perilla, also known as shiso (a herb in the mint family), followed by partial dehydration (Japanese Pat. Pub. No. 2005-160304 A).

Japanese Pat. Pub. No. 2011-193849 A provides a novel method of removing ammonia odor from urea-containing fish. The method involves the steps of shredding the fish, immersing it in a saline solution, steaming the fish, soaking the fish in raw soybean milk or the raw juice of vine plants within the Dioscoreaceae family, followed by a final steaming of the fish. The major technical innovation of this method is that raw soybean milk and vine plants within the Dioscoreaceae family contain urease enzyme in elevated amounts, which is able to chemically neutralize urea in the flesh of urea-containing fish. However, this method has the limitations of requiring that the fish be shredded as well as cooked twice, which would likely result in a lower quality final product and reduced consumer acceptability during consumption. Additionally, the additives may impart off-flavors that consumers may not enjoy.

Other methods suggest that soaking the fish in water, or processing the fish by squeezing, mincing, foaming, heating, binding, roasting, seasoning, rolling, molding, brining, exposure to ultraviolet light, or partially dehydrating all contribute to the effect of deodorization and urea/ammonia removal (Japanese Pat. Pub. No. H0731420 A, Japanese Pat. Pub. No. 2000-217501 A, Japanese Pat. Pub. No. 2001-128648 A, Japanese Pat. Pub. No. 2001-157563 A, U.S. Pat. No. 4,234,610, Japanese Pat. Pub. No. 2001-095471 A, Japanese Pat. Pub. No. 2001-245637 A, Japanese Pat. Pub. No. 2004-154113 A).

### BRIEF SUMMARY OF THE INVENTION

The present invention discloses a composition and method of neutralizing ammonia odor and flavor from shark and other urea-containing fish. The overall objective of the present invention is to increase the consumer acceptability of said fish during consumption.

In an example of the present invention, shark meat, or meat from other urea-containing fish, is soaked in a refrigerated (4° C.) bath containing water (88.0%), dry skim milk solids (7.5%), dry buttermilk solids (2.5%), and salt (2.0%) for 18-20 minutes.

Said fish are urea-containing fish within the Chondrichthyes class, such as sharks, rays, skates, sawfish, and chimaeras.

In an example of the present invention, shark meat, or meat from other urea-containing fish, prior to being placed in the bath, is processed to remove cartilage, skin, bone, and other unsavory parts of the fish which may otherwise result in decreased consumer acceptability during consumption.

The flesh of the fish may be sliced into strips with a thickness not exceeding 6.4 centimeters, which will aid in the complete absorption of the treatment ingredients.

The water fraction of the treatment bath dilutes and transports the dry skim milk solids, dry buttermilk solids, and salt into and out of the flesh of the fish during soaking, thusly allowing the ingredients to come into contact with the ammonia and other nitrogenous compounds present within the fish. Additionally, the water extracts some of the nitrogenous compounds during soaking, and is absorbed into the flesh of the fish, which reduces the amount of these compounds by mass difference.

The dry skim milk solids bind with and extract ammonia and other nitrogenous compounds through hydrogen bonding interactions between the milk protein (casein) and the nitrogenous compounds present within the flesh of the fish.

Similarly, the dry buttermilk solids also to extract ammonia and other nitrogenous compounds through casein-ammonia hydrogen bonding interactions. The dry buttermilk solids have the added benefit of containing elevated amounts of lactic acid (milk sugar) and fat compared to dry skim milk solids. The lactic acid present within the dry milk solids neutralizes ammonia and other nitrogenous compounds through chemical breakdown interactions. The fat content of the dry milk solids masks off-odors and flavors associated with the ammonia content of shark and other urea-containing fish.

The salt fraction extracts ammonia and other nitrogenous compounds through hydrogen bonding interactions between the salt ions and the nitrogenous compounds present within the flesh of the fish. Additionally, salt has the ability to increase moisture absorption into the flesh, which aids the effectiveness of the other added ingredients and reduces the ammonia content by mass difference. Furthermore, salt increases consumer acceptability due to an increase in perceived juiciness and the stimulation of salivary glands during consumption.

In an example of the present invention, the temperature of the treatment bath is at refrigerated temperature (for example 4° C.) to prevent the growth of pathogenic and spoilage microorganisms.

In an example of the invention, the fish are soaked for 18-20 minutes within the treatment bath, with 18 minutes being the minimal amount of time required for ingredient absorption and ammonia neutralization. Stirring, vibrating, or gentle agitation of the fish during soaking could be utilized to aid in ingredient absorption and ammonia neutralization.

### BRIEF DESCRIPTION OF THE SEVERAL VIEWS OF THE DRAWING

Reference will now be made, by way of example, to the accompanying drawing which shows an example embodiment of the present application, and in which:

FIG. 1 is block diagram illustrating an example method of neutralizing ammonia odor and flavor from shark fish.

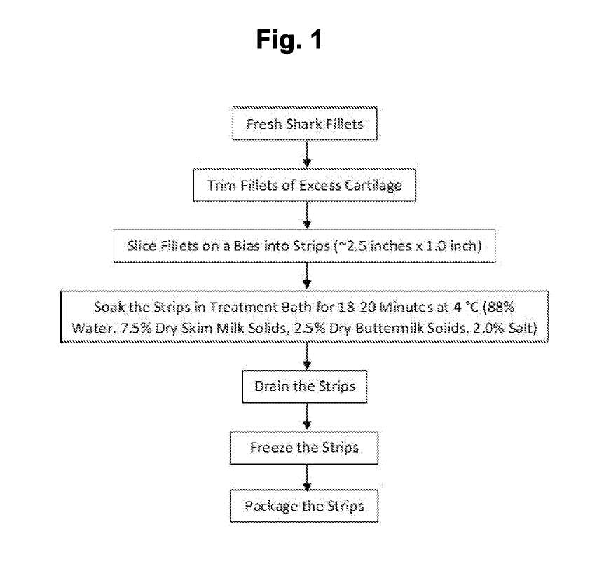

### DETAILED DESCRIPTION OF THE INVENTION

The U.S. provisional patent application Ser. No. 62/459,105 filed Feb. 15, 2017 is incorporated herein by reference.

The following detailed description and appended drawing describe and illustrate various exemplary embodiments of the invention. The description and drawing serve to enable one skilled in the art to make and use the invention, and are not intended to limit the scope of the invention in any manner. In respect of the methods disclosed, the steps presented are exemplary in nature, and thus, the order of the steps is not necessary or critical.

In some example implementations, shark, or other urea-containing fish, could be soaked in their whole form or processed into a variety of different shapes or conformations. For example, freshly caught shark could be minimally processed by simply heading and gutting the fish, and subsequently soaking in the treatment bath. Alternatively, shark could be processed into minced meat and subsequently soaked in the treatment bath.

In some example implementations, the treatment bath soak ingredients (water, dry skim milk solids, dry buttermilk solids, salt) may be utilized within the treatment bath at varying concentrations. Additionally, any one or two of the soak ingredients may be excluded completely from the treatment bath. For example, the treatment bath could be comprised of 80.0% water and 20.0% dry buttermilk solids to achieve the same effect as described in the example embodiment of the present invention comprising water (88.0%), dry skim milk solids (7.5%), dry buttermilk solids (2.5%), and salt (2.0%). While this specific composition exhibited desirable results, it should be understood that all conceivable variations in the relative compositions of elements of the composition are contemplated by the invention as claimed, even though such alternative relative compositions are not explicitly noted herein.

In some example implementations, the soak ingredients may be substituted with other milk or salt ingredients. For example, the salt may be selected from an edible salt which contains a sodium, potassium, calcium, aluminum, iron, magnesium, ammonium, or pyridinium cation; the milk ingredients may be selected from any mammalian milk or milk product. Alternately, for example, the salt fraction could be replaced with sodium tripolyphosphate or monosodium glutamate, both of which are effective salt substitutes and flavor enhancers within food products.

In some example implementations of the invention, the temperature of the treatment bath may be lower or higher than the example refrigerated temperature (4° C.), although not less than the temperature required to freeze the treatment bath or greater than the temperature required to cook the ingredients (40° C.). For example, the treatment bath could be cooled using ice, which would reduce the temperature to approximately 0° C. Alternatively, the fish could be soaked at room temperature (21° C.) in a pasteurized treatment bath.

In some example implementations, the fish may be soaked for less than or greater than 18-20 minutes. For example, the fish could be soaked for 5-10 minutes in a pressurized chamber, which would increase the rate of ingredient absorption and ammonia extraction/neutralization. Alternatively, the fish could be packaged with the treatment bath, which would result in the fish being soaked for an extended period of time throughout distribution.

The embodiments and example implementations of the present disclosure described above are intended to be examples only. The present disclosure may be embodied in other specific forms. Alterations, modifications and variations to the disclosure may be made without departing from the intended scope of the present disclosure. While the compositions and processes disclosed and shown herein may comprise a specific number of elements/components, the compositions, processes could be modified to include additional or fewer of such elements/components. Selected features from one or more of the above-described embodiments/implementations may be combined to create alternative embodiments not explicitly described. All values and sub-ranges within disclosed ranges are also included as well as any variations beyond disclosed ranges. The subject matter described herein intends to cover and embrace all suitable changes in technology. Persons skilled in the art will appreciate that the compositions and process parameters may be adjusted where desired.

In accordance with the provisions of the patent statutes, the invention has been described in what is considered to represent its preferred embodiment. However, it should be noted that the invention can be practiced otherwise than as specifically illustrated and described without departing from its spirit or scope.

1. A method of neutralizing ammonia odor and flavor from urea-containing fish, comprising the following step: placing the urea-containing fish in a bath containing water, dry skim milk solids, dry buttermilk solids, and salt.

2. The method according to claim 1 wherein the bath contains by weight the water at approximately 88.0%, the dry skim milk solids at approximately 7.5%, the dry buttermilk solids at approximately 2.5%, and the salt at approximately 2.0%.

3. The method according to claim 2 including refrigerating the bath and the fish in the bath at 4° C.

4. The method according to claim 2 including placing the fish in the bath for approximately 18-20 minutes.

5. The method according to claim 1 wherein the fish are urea-containing fish within a Chondrichthyes class.

6. The method according to claim 1 including selecting at least one of sharks, rays, skates, sawfish, and chimaeras as the fish.

7. The method according to claim 1 including, prior to placing the fish in the bath, processing the fish to remove cartilage, skin, bone, and other unsavory parts of the fish.

8. The method according to claim 1 including, prior to placing the fish in the bath, processing the fish to remove cartilage, skin, bone, and other unsavory parts of the fish, then cutting the fish into strips not exceeding 6.4 centimeters thick.

9. The method according to claim 1 including packaging the fish within the bath, whereby the fish are soaked for an extended period of time throughout distribution of the fish.

10. The method according to claim 1 including maintaining a temperature of the bath in a range from as low as 0° C. to as high as 40° C.

11. A method of neutralizing ammonia odor and flavor from urea-containing fish, comprising the following step: placing the urea-containing fish in a bath containing ingredients of at least two of water, dry skim milk solids, dry buttermilk solids and salt.

12. The method according to claim 11 wherein at least one of the ingredients is substituted with a different milk ingredient or a different salt ingredient.

13. The method according to claim 11 wherein the salt is an edible salt that contains at least one of sodium, potassium, calcium, aluminum, iron, magnesium, ammonium, and pyridinium cation.

14. The method according to claim 11 including soaking the fish within the bath for less than 18 minutes or greater than 20 minutes.

15. A composition for reducing unsavory ammonia odor and flavor from urea-containing fish, the composition comprising: at least two of water, dry skim milk solids, dry buttermilk solids, and salt.

16. The composition according to claim 15 containing the water at a percentage composition of approximately 88.0%, the dry skim milk solids at a percentage composition of approximately 7.5%, the dry buttermilk solids at a percentage composition of approximately 2.5%, and the salt at a percentage composition of approximately 2.0%.

17. The composition according to claim 15 wherein the salt is an edible salt that contains at least one of a sodium, potassium, calcium, aluminum, iron, magnesium, ammonium, and pyridinium cation.

18. The composition according to claim 15 wherein at least one of the dry skim milk solids and the dry buttermilk solids is selected from a mammalian milk or a milk product.
